(19) United States
(12) Patent Application Publication
(10) Pub. No.: US 2006/0161164 A1
(43) Pub. Date: Jul. 20, 2006

(54) Auxiliary bone reinforcement structure

(76) Inventors: Shiuh-Lin Hwang, Kaohsiung (TW); Wen-Hwa Kuo, Taipei (TW)

Correspondence Address: HERSHKOVITZ & ASSOCIATES, 1725 I STREET NW, SUITE 300, WASHINGTON, DC, 20006, US

(21) Appl. No.: 11/034,778

(22) Filed: Jan. 14, 2005

(51) Int. Cl. A61F 5/00

(52) U.S. Cl. 606/86

(57) ABSTRACT

An auxiliary bone reinforcement structure includes a deformable hollow body having multiple bosses formed on an outer periphery of the hollow body and a connection portion, a connector detachably connected to the connection portion and having an annular groove defined in an outer periphery of the connector and an injector detachably connected to the connector for injection of a medicine into the hollow body whereby the hollow body is deformed after the medicine is injected into the hollow body and thus able to stuff a hole in the bone.

### BACKGROUND OF THE INVENTION

1. Field of the Invention

The present invention relates to an auxiliary bone reinforcement structure, and more particularly to an auxiliary bone reinforcement structure that can effectively strengthen the bone without endangering surrounding tissues.

2. Description of Related Art

Patients with osteoporosis normally suffer from the loss of organic substances in the bone or are unable to maintain a certain amount of organic substances so that due to the decrease of bone density, these patients easily suffer from fractures. To improve the conditions, food with lots of calcium is the best supplement to enrich the organic substances in the bone. However, using food to improve the bone conditions takes a long time to see the result. To those who need immediate medical attendance to improve the bone conditions, food seems not adequate. In order to enhance the bone conditions in a relative short period of time, a new approach is developed. Doctors are able to inject a kind of medicine into the bone and wait for the solidification of the medicine. Thereafter, the bone structure is strengthened in a short period of time. However, because there is no limitation to the injected medicine inside the bone after the medicine is injected into the bone, the medicine may flow before solidification to other areas and thus causes side effects to such as damage to veins and other organs.

To overcome the shortcomings, the present invention tends to provide an improved auxiliary bone reinforcement structure to mitigate the aforementioned problems.

### SUMMARY OF THE INVENTION

The primary objective of the present invention is to provide an auxiliary bone reinforcement structure to effectively support the bone so as to enhance the bone structure within a short period of time.

In order to accomplish the objective, the auxiliary bone reinforcement structure of the present invention includes a body, a connector and an injector.

The body has a star-fruit-like hollow cylinder with a connection portion formed on an end of the cylinder. The connector has a first end detachably connected to the connection portion and a second end. The injector is composed of an outer tube, an inner tube and a plunger.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view of the auxiliary bone reinforcement structure of the present invention;

FIG. 2 is a cross sectional view of the cylinder of the auxiliary bone reinforcement structure of the present invention;

FIG. 3 is a cross sectional view showing the assembled auxiliary bone reinforcement structure of the present invention;

FIG. 4 is a schematic view showing that the cylinder is inserted into a hole defined in a bone; and

FIG. 5 is a schematic view showing that after the medicine is injected into the cylinder and solidified, the cylinder is able to fill the hole in the bone so as to provide support to the bone.

### DETAILED DESCRIPTION OF THE PREFERRED EMBODIMENT

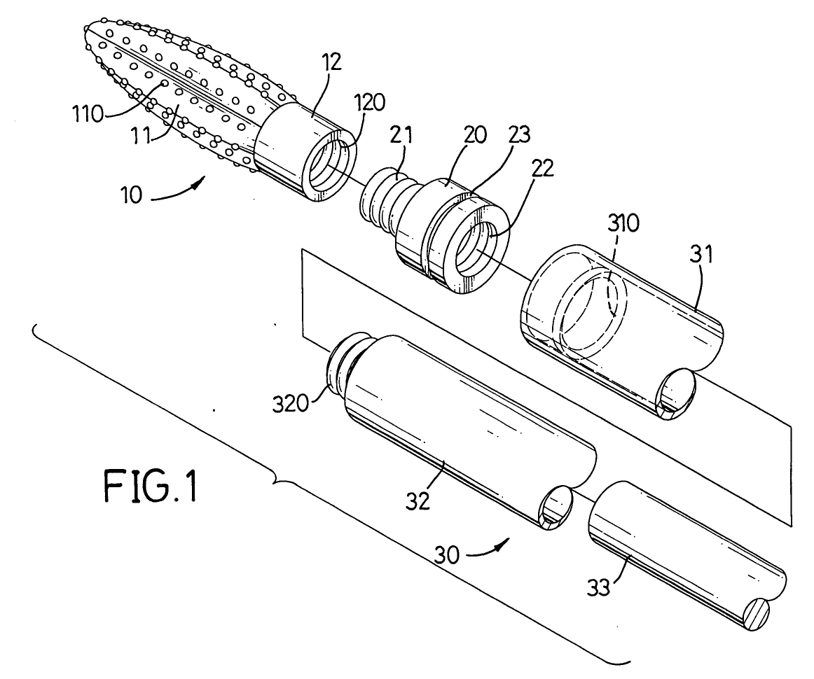

With reference to FIG. 1, the auxiliary bone reinforcement structure in accordance with the present invention includes a body (10), a connector (20) and an injector (30).

The body (10) has a first end with a hollow cylinder (11) having multiple bosses (110) formed on an outer periphery of the cylinder (11) and a second end provided with a connection portion (12) and having a first connecting threading (120) defined in an inner face of the connection portion (12). Preferably the body (10) is made of silicone so that the body (10) has good elasticity and deformation ability.

The connector (20) has a first end with a second connecting threading (21) formed on an outer periphery of the first end of the connector (20) to correspond to the first connecting threading (120) of the connection portion (12), a third connecting threading (22) formed on an inner face of a second end thereof and an annular groove (23) defined in the outer periphery of the connector (20).

The injector (30) includes an outer tube (31) having a flange (310) formed on an inner face of the outer tube (31) to correspond to the annular groove (23) of the connector (20), an inner tube (32) received in the outer tube (31) and having a fourth connecting threading (320) formed on a first end thereof to correspond to the third connecting threading (22) of the connector (20) and an open end defined in a second end of the inner tube (32), and a plunger (33) slidably received in the inner tube (32).

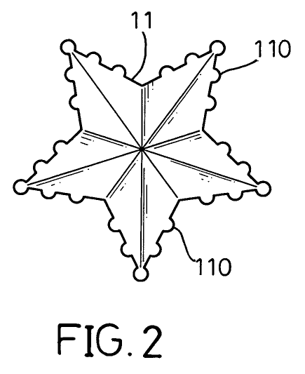

With reference to FIG. 2, it is noted that the cylinder (11) is hollow and the bosses (110) formed on the outer periphery of the cylinder (11) cause the cylinder (11) to be a star-fruit-like body.

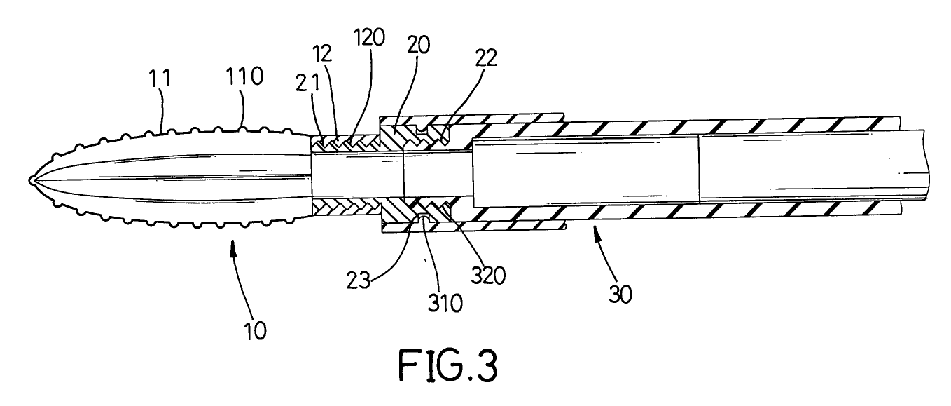

With reference to FIG. 3, when the auxiliary bone reinforcement structure of the present invention is assembled, the second connecting threading (21) is threadingly connected to the first connecting threading (120) of the body (10). The second end of the connector (20) is inserted into the outer tube (31) to have the flange (310) to be received in the corresponding annular groove (23). After extending through the outer tube (31), the fourth connecting threading (320) of the inner tube (32) is threadingly connected to the third connecting threading (22) to combine the connector (20) and the inner tube (32). Then the plunger (33) is inserted into the inner tube (32).

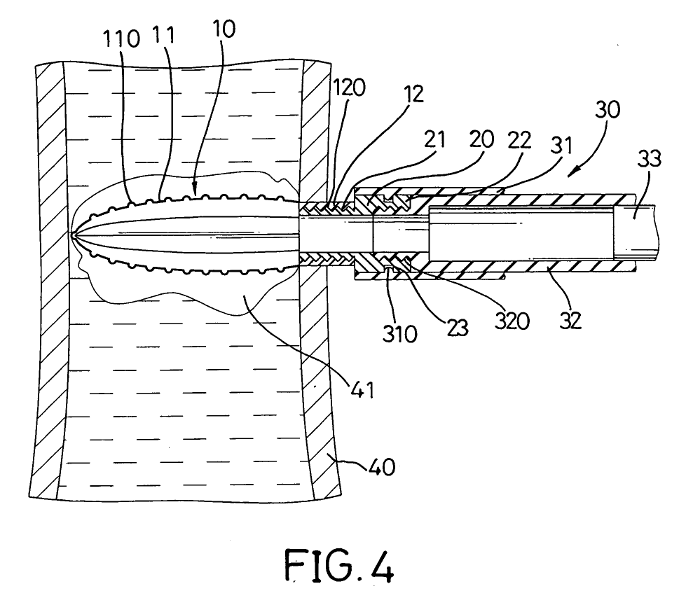

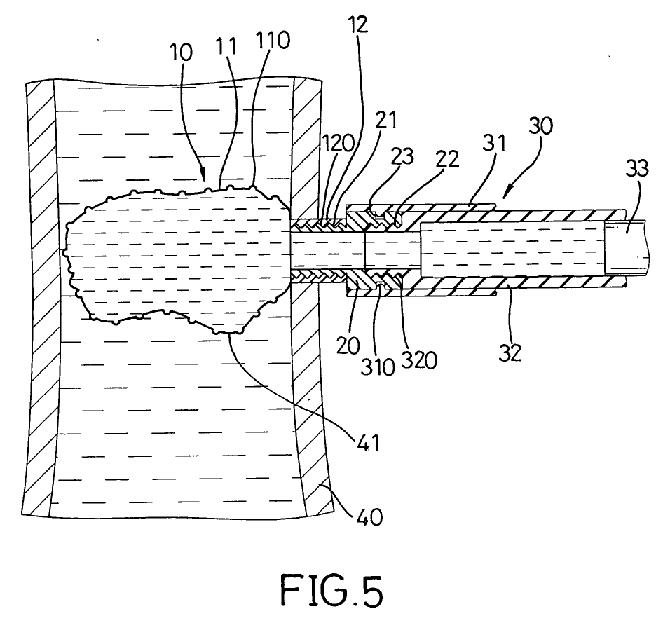

With reference to FIGS. 4 and 5, when the auxiliary bone reinforcement structure of the present invention is in application, a hole (41) is first defined in the bone (40). Then the cylinder (11) is inserted into the hole (41). After the cylinder (11) is rested inside the hole (41), medicine received in the inner tube (32) is injected by forward movement of the plunger (33) relative to the outer tube (31). Because the connector (20) is hollow, The medicine in the inner tube (32) is able to be injected to the cylinder (11) to expand the cylinder (11). Due to the elasticity and ability to deform, the expanded cylinder (11) is able to stuff the hole (41) in the bone (40).

Thereafter, when the medicine in the cylinder (11) has hardened, the bone has great support from the hardened medicine. Removal of the connector (20) as well as the injector (30) completes the bone treatment process.

The star-fruit-like configuration and the multiple bosses (110) are found to be particularly effective in forming a non-slip grip with the surfaces when they are in contact, such that the patient does not suffer any discomfort from movement of the cylinder within the cavity wound.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. An auxiliary bone reinforcement structure comprising:
a deformable hollow body having a first end with multiple bosses formed on an outer periphery of the hollow body and a connection portion formed on a second end of the hollow body;
a connector detachably connected to the connection portion and having an annular groove defined in an outer periphery of the connector; and
an injector detachably connected to the connector for injection of a medicine into the hollow body whereby the hollow body is deformed after the medicine is injected into the hollow body and thus able to stuff a hole in the bone.

2. The auxiliary bone reinforcement structure as claimed in claim 1, wherein the hollow body has a cylinder formed on the first end and the bosses are formed on an outer periphery of the cylinder to become a star-fruit-like cylinder.

3. The auxiliary bone reinforcement structure as claimed in claim 1, wherein the connection portion has a first connection structure and the connector has a second connection structure to correspond to the first connection structure to allow the connector to be detachably connected to the connection portion of the hollow body.

4. The auxiliary bone reinforcement structure as claimed in claim 2, wherein the connection portion has a first connection structure and the connector has a second connection structure to correspond to the first connection structure to allow the connector to be detachably connected to the connection portion of the hollow body.

5. The auxiliary bone reinforcement structure as claimed in claim 1, wherein the injector has an outer tube, an inner tube extending through the outer tube and a plunger slidably received in the inner tube, the outer tube has a flange formed on an inner face thereof to correspond to and be received in the annular groove of the connector, the connector has a third connection structure formed relative to the second connection structure to correspond to a fourth connection structure which is formed on a first end of the inner tube such that the inner tube is able to be detachably connected to the connector after the inner tube is extended through the outer tube such that movement of the plunger toward the hollow body is able to force the medicine from inside the inner tube to the hollow body.

6. The auxiliary bone reinforcement structure as claimed in claim 2, wherein the injector has an outer tube, an inner tube extending through the outer tube and a plunger slidably received in the inner tube, the outer tube has a flange formed on an inner face thereof to correspond to and received in the annular groove of the connector, the connector has a third connection structure formed relative to the second connection structure to correspond to a fourth connection structure which is formed on a first end of the inner tube such that the inner tube is able to be detachably connected to the connector after the inner tube is extended through the outer tube such that movement of the plunger toward the hollow body is able to force the medicine from inside the inner tube to the hollow body.

7. The auxiliary bone reinforcement structure as claimed in claim 3, wherein the injector has an outer tube, an inner tube extending through the outer tube and a plunger slidably received in the inner tube, the outer tube has a flange formed on an inner face thereof to correspond to and be received in the annular groove of the connector, the connector has a third connection structure formed relative to the second connection structure to correspond to a fourth connection structure which is formed on a first end of the inner tube such that the inner tube is able to be detachably connected to the connector after the inner tube is extended through the outer tube such that movement of the plunger toward the hollow body is able to force the medicine from inside the inner tube to the hollow body.

8. The auxiliary bone reinforcement structure as claimed in claim 4, wherein the injector has an outer tube, an inner tube extending through the outer tube and a plunger slidably received in the inner tube, the outer tube has a flange formed on an inner face thereof to correspond to and be received in the annular groove of the connector, the connector has a third connection structure formed relative to the second connection structure to correspond to a fourth connection structure which is formed on a first end of the inner tube such that the inner tube is able to be detachably connected to the connector after the inner tube is extended through the outer tube such that movement of the plunger toward the hollow body is able to force the medicine from inside the inner tube to the hollow body.
